SEQUENCE LISTING (1 sequence)

SEQ ID NO: 1 - protein, 467 aa, Artificial Sequence
  Fusion
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2020/0323958 A1
(43) Pub. Date: Oct. 15, 2020

(54) METHODS FOR PRODUCING PROTEIN PRODUCTS

(71) Applicant: AMGEN INC., Thousand Oaks, CA (US)

(72) Inventor: Eliyahu Kraus, Sutton, MA (US)

(73) Assignee: AMGEN INC., Thousand Oaks, CA (US)

(21) Appl. No.: 16/763,515

(22) PCT Filed: Nov. 5, 2018
(86) PCT No.: PCT/US18/59213

§ 371 Date: May 12, 2020

Related U.S. Application Data
(60) Provisional application No. 62/585,397, filed on Nov. 13, 2017.

(51) Int. Cl. A61K 38/17; C12M 1/34; A61K 47/02; A61K 47/26

(52) U.S. Cl. CPC A61K 38/1793; A61K 47/26; A61K 47/02; C12M 41/12

(57) ABSTRACT

The invention pertains to methods of improving the yield and preserving the quality of recombinantly produced proteins and polypeptides in a production bioreactor process by manipulating or controlling the temperature of a bioreactor process that precedes the ultimate production bioreactor process. In particular embodiments the invention provides such methods as well as recombinant proteins and polypeptides produced using such methods, formulations thereof, and methods of treating patients using such proteins, polypeptides, and formulations.

### CROSS-REFERENCE TO RELATED APPLICATION

This application claims the benefit of U.S. Provisional Application No. 62/585,397 filed Nov. 13, 2017, which is incorporated in its entirety by reference herein.

### FIELD OF THE INVENTION

The present invention relates generally to the production of recombinant proteins in bioreactors.

### BACKGROUND OF THE INVENTION

Clinical manufacture of therapeutic proteins is an expensive, large scale endeavor. Maintaining cell growth and viability throughout the cell culture process is very important. As the demand for greater quantities of therapeutic recombinant proteins increases, positive increases in protein production, cell growth and viability are sought out by implementing new methods to improve cell development, media optimization and process control parameters, and to intensify harvest and purification processes.

The production of recombinant polypeptides for use as pharmaceutical therapeutics typically utilizes a drug substance cell culture process that consists of a series of scale-up and expansion phases designed to generate sufficient cell mass for inoculation of a production bioreactor. The cell culture process is initiated by thawing a vial from the working cell bank (WCB) and expanding the culture by using, for example, a series of shake flasks, culture bags, and/or expansion seed bioreactors (e.g., N-3, N-2, wherein the number indicates how many steps antecedent a bioreactor is from N, the final, or production, bioreactor), or the like. Following growth in the seed culture bioreactors, the culture is transferred to the N-1 bioreactor, which can be, for example, a perfusion bioreactor. In an N-1 perfusion bioreactor, the culture is perfused with fresh medium in order to generate sufficient cell densities for inoculation of the final cultivation step, the production bioreactor (N). The production bioreactor is operated to maximize the efficient production of the recombinant polypeptide.

Most of the efforts to date to maximize the efficient production of recombinant polypeptides have involved manipulating the N production bioreactor run. A relationship between N (production) bioreactor temperature and titer has been demonstrated for etanercept and for other products. The cause of the increased titer when N bioreactor temperature is optimized is due to both an increase in specific productivity (the amount of protein produced by cells over time, as measured, e.g., by picogram protein/cell/day) as well as an increase in cell growth. Similarly, a relationship between titer and each of N bioreactor pH, initial viable cell density (iVCD) and duration parameters has been shown. While titer increases induced by optimizing iVCD and duration tend to be due to an increase or maintenance in cell mass, titer increases due to pH can be due to increased cell mass and/or increased specific productivities.

However, to date no direct relationship has been shown between N-1 bioreactor parameters and N bioreactor titer.

ENBREL® (etanercept; Immunex Inc., Thousand Oaks, Calif.) is a covalently dimerized form of recombinant human tumor necrosis factor receptor (TNFR): Fc fusion protein produced in genetically engineered Chinese hamster ovary (CHO) cells.

### SUMMARY OF THE INVENTION

The present application reports for the first time the unexpected observation that the temperature of an N-1 perfusion bioreactor run can be controlled or manipulated to increase the productivity of a subsequent N production bioreactor in a way that is independent of the titer of cells that is used to inoculate the N production bioreactor. Accordingly, the present invention provides methods and devices for improving the N production bioreactor run titer of a recombinant polypeptide by manipulating or controlling the temperature of an N-1 perfusion bioreactor run that precedes it.

The present invention also provides a composition resulting from an N production bioreactor run that was preceded by an N-1 perfusion bioreactor with a manipulated or controlled temperature as described herein. In a specific embodiment, the present invention provides an etanercept product made according to the production techniques of the present invention.

### BRIEF DESCRIPTION OF THE DRAWINGS

FIG. 1 provides a graph demonstrating the relationship between N-1 bioreactor temperature and N bioreactor titer level residuals.

FIG. 2 provides a graph demonstrating the relationship between N-1 bioreactor culture duration and N bioreactor titer level residuals.

FIG. 3 provides a graph demonstrating the relationship between N-1 bioreactor pH and N bioreactor titer level residuals.

FIG. 4 provides a graph demonstrating the relationship between N-1 bioreactor iVCD and N bioreactor titer level residuals.

### DETAILED DESCRIPTION OF THE INVENTION

The present invention is based in part on the novel and unexpected demonstration that the N production bioreactor titer of a recombinant polypeptide can be increased by controlling the temperature at which the N-1 perfusion bioreactor is run as disclosed herein for the first time.

While the terminology used in this application is standard within the art, definitions of certain terms are provided herein to assure clarity and definiteness to the meaning of the claims. Units, prefixes, and symbols may be denoted in their SI accepted form. Numeric ranges recited herein are inclusive of the numbers defining the range and include and are supportive of each integer within the defined range. Unless otherwise noted, the terms “a” or “an” are to be construed as meaning “at least one of”. The section headings used herein are for organizational purposes only and are not to be construed as limiting the subject matter described. The methods and techniques described herein are generally performed according to conventional methods well known in the art and as described in various general and more specific references that are cited and discussed throughout the present specification unless otherwise indicated. See, e.g., Sambrook et al. Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990). All documents, or portions of documents, cited in this application, including but not limited to patents, patent applications, articles, books, and treatises, are hereby expressly incorporated by reference.

As used herein “peptide,” “polypeptide” and “protein” are used interchangeably throughout and refer to a molecule comprising two or more amino acid residues joined to each other by peptide bonds. Peptides, polypeptides and proteins are also inclusive of modifications including, but not limited to, glycosylation, lipid attachment, sulfation, gamma-carboxylation of glutamic acid residues, hydroxylation and ADP-ribosylation. Polypeptides can be of scientific or commercial interest, including protein-based drugs. Polypeptides include, among other things, antibodies, fusion proteins, and cytokines. Peptides, polypeptides and proteins are produced by recombinant animal cell lines using cell culture methods and may be referred to as “recombinant peptide”, “recombinant polypeptide” and “recombinant protein”. The expressed protein(s) may be produced intracellularly or secreted into the culture medium from which it can be recovered and/or collected.

Examples of polypeptides that can be produced with the methods of the invention include proteins comprising amino acid sequences identical to or substantially similar to all or part of one of the following proteins: tumor necrosis factor (TNF), flt3 ligand (WO 94/28391), erythropoeitin, thrombopoeitin, calcitonin, IL-2, angiopoietin-2 (Maisonpierre et al. (1997), Science 277(5322): 55-60), ligand for receptor activator of NF-kappa B (RANKL, WO 01/36637), tumor necrosis factor (TNF)-related apoptosis-inducing ligand (TRAIL, WO 97/01633), thymic stroma-derived lymphopoietin, granulocyte colony stimulating factor, granulocyte-macrophage colony stimulating factor (GM-CSF, Australian Patent No. 588819), mast cell growth factor, stem cell growth factor (U.S. Pat. No. 6,204,363), epidermal growth factor, keratinocyte growth factor, megakaryote growth and development factor, RANTES, human fibrinogen-like 2 protein (FGL2; NCBI accession no. NM-00682; Rüegg and Pytela (1995), Gene 160:257-62) growth hormone, insulin, insulinotropin, insulin-like growth factors, parathyroid hormone, interferons including α-interferons, γ-interferon, and consensus interferons (U.S. Pat. Nos. 4,695,623 and 4,897,471), nerve growth factor, brain-derived neurotrophic factor, synaptotagmin-like proteins (SLP 1-5), neurotrophin-3, glucagon, interleukins, colony stimulating factors, lymphotoxin-β, leukemia inhibitory factor, and oncostatin-M. Descriptions of proteins that can be produced according to the inventive methods may be found in, for example, Human Cytokines: Handbook for Basic and Clinical Research, all volumes (Aggarwal and Gutterman, eds. Blackwell Sciences, Cambridge, Mass., 1998); Growth Factors: A Practical Approach (McKay and Leigh, eds., Oxford University Press Inc., New York, 1993); and The Cytokine Handbook, Vols. 1 and 2 (Thompson and Lotze eds., Academic Press, San Diego, Calif., 2003).

Additionally the methods of the invention would be useful to produce proteins comprising all or part of the amino acid sequence of a receptor for any of the above-mentioned proteins, an antagonist to such a receptor or any of the above-mentioned proteins, and/or proteins substantially similar to such receptors or antagonists. These receptors and antagonists include: both forms of tumor necrosis factor receptor (TNFR, referred to as p55 and p75, U.S. Pat. Nos. 5,395,760 and 5,610,279), Interleukin-1 (IL-1) receptors (types I and II; EP Patent No. 0460846, U.S. Pat. Nos. 4,968,607, and 5,767,064), IL-1 receptor antagonists (U.S. Pat. No. 6,337,072), IL-1 antagonists or inhibitors (U.S. Pat. Nos. 5,981,713, 6,096,728, and 5,075,222) IL-2 receptors, IL-4 receptors (EP Patent No. 0 367 566 and U.S. Pat. No. 5,856,296), IL-15 receptors, IL-17 receptors, IL-18 receptors, Fc receptors, granulocyte-macrophage colony stimulating factor receptor, granulocyte colony stimulating factor receptor, receptors for oncostatin-M and leukemia inhibitory factor, receptor activator of NF-kappa B (RANK, WO 01/36637 and U.S. Pat. No. 6,271,349), osteoprotegerin (U.S. Pat. No. 6,015,938), receptors for TRAIL (including TRAIL receptors 1, 2, 3, and 4), and receptors that comprise death domains, such as Fas or Apoptosis-Inducing Receptor (AIR).

Other proteins that can be produced using the invention include proteins comprising all or part of the amino acid sequences of differentiation antigens (referred to as CD proteins) or their ligands or proteins substantially similar to either of these. Such antigens are disclosed in Leukocyte Typing VI (Proceedings of the VIth International Workshop and Conference, Kishimoto, Kikutani et al., eds., Kobe, Japan, 1996). Similar CD proteins are disclosed in subsequent workshops. Examples of such antigens include CD22, CD27, CD30, CD39, CD40, and ligands thereto (CD27 ligand, CD30 ligand, etc.). Several of the CD antigens are members of the TNF receptor family, which also includes 41BB and OX40. The ligands are often members of the TNF family, as are 41BB ligand and OX40 ligand.

Enzymatically active proteins or their ligands can also be produced using the invention. Examples include proteins comprising all or part of one of the following proteins or their ligands or a protein substantially similar to one of these: a disintegrin and metalloproteinase domain family members including TNF-alpha Converting Enzyme, various kinases, glucocerebrosidase, superoxide dismutase, tissue plasminogen activator, Factor VIII, Factor IX, apolipoprotein E, apolipoprotein A-I, globins, an IL-2 antagonist, alpha-1 antitrypsin, ligands for any of the above-mentioned enzymes, and numerous other enzymes and their ligands.

The term “antibody” includes reference to both glycosylated and non-glycosylated immunoglobulins of any isotype or subclass or to an antigen-binding region thereof that competes with the intact antibody for specific binding, unless otherwise specified, including human, humanized, chimeric, multi-specific, monoclonal, polyclonal, and oligomers or antigen binding fragments thereof. Also included are proteins having an antigen binding fragment or region such as Fab, Fab′, F(ab′)2, Fv, diabodies, Fd, dAb, maxibodies, single chain antibody molecules, complementarity determining region (CDR) fragments, scFv, diabodies, triabodies, tetrabodies and polypeptides that contain at least a portion of an immunoglobulin that is sufficient to confer specific antigen binding to a target polypeptide. The term “antibody” is inclusive of, but not limited to, those that are prepared, expressed, created or isolated by recombinant means, such as antibodies isolated from a host cell transfected to express the antibody.

Examples of antibodies include, but are not limited to, those that recognize any one or a combination of proteins including, but not limited to, the above-mentioned proteins and/or the following antigens: CD2, CD3, CD4, CD8, CD11a, CD14, CD18, CD20, CD22, CD23, CD25, CD33, CD40, CD44, CD52, CD80 (B7.1), CD86 (B7.2), CD147, IL-la, IL-1(3, IL-2, IL-3, IL-7, IL-4, IL-5, IL-8, IL-10, IL-2 receptor, IL-4 receptor, IL-6 receptor, IL-13 receptor, IL-18 receptor subunits, FGL2, PDGF-β and analogs thereof (see U.S. Pat. Nos. 5,272,064 and 5,149,792), VEGF, TGF, TGF-β2, TGF-β1, EGF receptor (see U.S. Pat. No. 6,235,883) VEGF receptor, hepatocyte growth factor, osteoprotegerin ligand, interferon gamma, B lymphocyte stimulator (BlyS, also known as BAFF, THANK, TALL-1, and zTNF4; see Do and Chen-Kiang (2002), Cytokine Growth Factor Rev. 13(1): 19-25), C5 complement, IgE, tumor antigen CA125, tumor antigen MUC1, PEM antigen, LCG (which is a gene product that is expressed in association with lung cancer), HER-2, HER-3, a tumor-associated glycoprotein TAG-72, the SK-1 antigen, tumor-associated epitopes that are present in elevated levels in the sera of patients with colon and/or pancreatic cancer, cancer-associated epitopes or proteins expressed on breast, colon, squamous cell, prostate, pancreatic, lung, and/or kidney cancer cells and/or on melanoma, glioma, or neuroblastoma cells, the necrotic core of a tumor, integrin alpha 4 beta 7, the integrin VLA-4, B2 integrins, TRAIL receptors 1, 2, 3, and 4, RANK, RANK ligand, TNF-α, the adhesion molecule VAP-1, epithelial cell adhesion molecule (EpCAM), intercellular adhesion molecule-3 (ICAM-3), leukointegrin adhesin, the platelet glycoprotein gp IIb/IIIa, cardiac myosin heavy chain, parathyroid hormone, rNAPc2 (which is an inhibitor of factor VIIa-tissue factor), MHC I, carcinoembryonic antigen (CEA), alpha-fetoprotein (AFP), tumor necrosis factor (TNF), CTLA-4 (which is a cytotoxic T lymphocyte-associated antigen), Fc-γ-1 receptor, HLA-DR 10 beta, HLA-DR antigen, sclerostin, L-selectin, Respiratory Syncitial Virus, human immunodeficiency virus (HIV), hepatitis B virus (HBV), Streptococcus mutans, and Staphlycoccus aureus. Specific examples of known antibodies which can be produced using the methods of the invention include but are not limited to adalimumab, bevacizumab, infliximab, abciximab, alemtuzumab, bapineuzumab, basiliximab, belimumab, briakinumab, canakinumab, certolizumab pegol, cetuximab, conatumumab, denosumab, eculizumab, gemtuzumab ozogamicin, golimumab, ibritumomab tiuxetan, labetuzumab, mapatumumab, matuzumab, mepolizumab, motavizumab, muromonab-CD3, natalizumab, nimotuzumab, ofatumumab, omalizumab, oregovomab, palivizumab, panitumumab, pemtumomab, pertuzumab, ranibizumab, rituximab, rovelizumab, tocilizumab, tositumomab, trastuzumab, ustekinumab, vedolizomab, zalutumumab, and zanolimumab.

The invention can also be used to produce recombinant fusion proteins comprising, for example, any of the above-mentioned proteins. For example, recombinant fusion proteins comprising one of the above-mentioned proteins plus a multimerization domain, such as a leucine zipper, a coiled coil, an Fc portion of an immunoglobulin, or a substantially similar protein, can be produced using the methods of the invention. See e.g. WO94/10308; Lovejoy et al. (1993), Science 259:1288-1293; Harbury et al. (1993), Science 262:1401-05; Harbury et al. (1994), Nature 371:80-83; Häkansson et al. (1999), Structure 7:255-64. Specifically included among such recombinant fusion proteins are proteins in which a portion of a receptor is fused to an Fc portion of an antibody such as etanercept (a p75 TNFR:Fc), and belatacept (CTLA4:Fc).

For the purposes of this invention, cell culture medium is a media suitable for growth of animal cells, such as mammalian cells, in in vitro cell culture. Cell culture media formulations are well known in the art. Typically, cell culture media are comprised of buffers, salts, carbohydrates, amino acids, vitamins and trace essential elements. The cell culture medium may or may not contain serum, peptone, and/or proteins. Various tissue culture media, including serum-free and defined culture media, are commercially available, for example, any one or a combination of the following cell culture media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series (JRH Biosciences, Lenexa, Kans.), among others. Cell culture media may be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters.

Cell culture media may be serum-free, protein-free, and/or peptone-free. “Serum-free” applies to a cell culture medium that does not contain animal sera, such as fetal bovine serum. “Protein-free” applies to cell culture media free from exogenously added protein, such as transferrin, protein growth factors IGF-1, or insulin. Protein-free media may or may not contain peptones. “Peptone-free” applies to cell culture media which contains no exogenous protein hydrolysates such as animal and/or plant protein hydrolysates. Eliminating serum and/or hydrolysates from cell culture media has the advantage of reducing lot to lot variability and enhancing processing steps, such as filtration. However, when serum and/or peptone are removed from the cell culture media, cell growth, viability and/or protein expression may be diminished or less than optimal. As such, serum-free and/or peptone-free cell culture medium may be highly enriched for amino acids, trace elements and the like. See, for example, U.S. Pat. Nos. 5,122,469 and 5,633,162. Although there are many media formulations, there is a need to develop defined media formulations that perform as well or preferably better than those containing animal sera and/or peptones.

Defined cell culture media formulations are complex, containing amino acids, inorganic salts, carbohydrates, lipids, vitamins, buffers and trace essential elements. Identifying the components that are necessary and beneficial to maintain a cell culture with desired characteristics is an on going task. Defined basal media formulations which are supplemented or enriched to meet the needs of a particular host cell or to meet desired performance parameters is one approach to developing defined media. Identifying those components and optimum concentrations that lead to improved cell growth, viability and protein production is an ongoing task.

By cell culture or “culture” is meant the growth and propagation of cells outside of a multicellular organism or tissue. Suitable culture conditions for mammalian cells are known in the art. See e.g. Animal cell culture: A Practical Approach, D. Rickwood, ed., Oxford University Press, New York (1992). Mammalian cells may be cultured in suspension or while attached to a solid substrate. Fluidized bed bioreactors, hollow fiber bioreactors, roller bottles, shake flasks, or stirred tank bioreactors, with or without microcarriers, and operated in a batch, fed batch, continuous, semi-continuous, or perfusion mode are available for mammalian cell culture.

Mammalian cells, such as CHO cells, may be cultured in small scale cultures, such as for example, in 100 ml containers having about 30 ml of media, 250 ml containers having about 80 to about 90 ml of media, 250 ml containers having about 150 to about 200 ml of media. Alternatively, the cultures can be large scale such as for example 1000 ml containers having about 300 to about 1000 ml of media, 3000 ml containers having about 500 ml to about 3000 ml of media, 8000 ml containers having about 2000 ml to about 8000 ml of media, and 15000 ml containers having about 4000 ml to about 15000 ml of media. Large scale cell cultures, such as for clinical manufacturing of protein therapeutics, are typically maintained for days, or even weeks, while the cells produce the desired protein(s).

During this time the culture can be supplemented with a concentrated feed medium containing components, such as nutrients and amino acids, which are consumed during the course of the production phase of the cell culture. Concentrated feed medium may be based on just about any cell culture media formulation. Such a concentrated feed medium can contain most of the components of the cell culture medium at, for example, about 5×, 6×, 7×, 8×, 9×, 19×, 12×, 14×, 16×, 20×, 30×, 50×, 100×, 200×, 400×, 600×, 800×, or even about 1000× of their normal amount. Concentrated feed media are often used in fed batch culture processes.

Fed batch culture is a widely-practiced culture method for large scale production of proteins from mammalian cells. See e.g. Chu and Robinson (2001), Current Opin. Biotechnol. 12: 180-87. A fed batch culture of mammalian cells is one in which the culture is fed, either continuously or periodically, with a concentrated feed medium containing nutrients. Feeding can occur on a predetermined schedule of, for example, every day, once every two days, once every three days, etc. The culture can be monitored for tyrosine, cystine and/or cysteine levels in the culture medium and can be adjusted through feedings of a concentrated tyrosine or tyrosine and cystine solution so as to keep tyrosine, cysteine and/or cystine within a desired range. When compared to a batch culture, in which no feeding occurs, a fed batch culture can produce greater amounts of protein. See e.g. U.S. Pat. No. 5,672,502.

The method according to the present invention may be used to improve the production of recombinant proteins in a drug substance productions process whereby cells are cultured in two or more distinct phases. For example, cells may be cultured first in one or more growth phases, under environmental conditions that maximize cell proliferation and viability, then transferred to a production phase, under conditions that maximize protein production. Each phase can be conducted in its own bioreactor vessel, or more than phase can be conducted in a common bioreactor vessel. In one such example, a growth phase and a production phase are conducted in the same bioreactor vessel. In a commercial process for production of a protein by mammalian cells, there are commonly multiple, for example, at least about 2, 3, 4, 5, 6, 7, 8, 9, or 10 growth phases that occur in different culture vessels preceding a final production phase. The method according to the present invention can be employed at least during the N-1 growth phase, although it may also be employed in a preceding growth phase. A production phase can be conducted at large scale. A large scale process can be conducted in a volume of at least about 100, 500, 1000, 2000, 3000, 5000, 7000, 8000, 10,000, 15,000, 20,000 liters. A growth phase may occur at a higher temperature than a production phase. For example, a growth phase may occur at a first temperature from about 35° C. to about 38° C., and a production phase may occur at a second temperature from about 29° C. to about 37° C., optionally from about 30° C. to about 36° C. or from about 30° C. to about 34° C. In addition, chemical inducers of protein production, such as, for example, caffeine, butyrate, and hexamethylene bisacetamide (HMBA), may be added at the same time as, before, and/or after a temperature shift. If inducers are added after a temperature shift, they can be added from one hour to five days after the temperature shift, optionally from one to two days after the temperature shift.

The cell lines (also referred to as “host cells”) used in the invention are genetically engineered to express a polypeptide of commercial or scientific interest. Cell lines are typically derived from a lineage arising from a primary culture that can be maintained in culture for an unlimited time. Genetically engineering the cell line involves transfecting, transforming or transducing the cells with a recombinant polynucleotide molecule, and/or otherwise altering (e.g., by homologous recombination and gene activation or fusion of a recombinant cell with a non-recombinant cell) so as to cause the host cell to express a desired recombinant polypeptide. Methods and vectors for genetically engineering cells and/or cell lines to express a polypeptide of interest are well known to those of skill in the art; for example, various techniques are illustrated in Current Protocols in Molecular Biology, Ausubel et al., eds. (Wiley & Sons, New York, 1988, and quarterly updates); Sambrook et al., Molecular Cloning: A Laboratory Manual (Cold Spring Laboratory Press, 1989); Kaufman, R. J., Large Scale Mammalian Cell Culture, 1990, pp. 15-69.

Animal cell lines are derived from cells whose progenitors were derived from a multi-cellular animal. One type of animal cell line is a mammalian cell line. A wide variety of mammalian cell lines suitable for growth in culture are available from the American Type Culture Collection (Manassas, Va.) and commercial vendors. Examples of cell lines commonly used in the industry include VERO, BHK, HeLa, CV1 (including Cos), MDCK, 293, 3T3, myeloma cell lines (e.g., NSO, NS1), PC12, WI38 cells, and Chinese hamster ovary (CHO) cells. CHO cells are widely used for the production of complex recombinant proteins, e.g. cytokines, clotting factors, and antibodies (Brasel et al. (1996), Blood 88:2004-2012; Kaufman et al. (1988), J. Biol Chem 263:6352-6362; McKinnon et al. (1991), J Mol Endocrinol 6:231-239; Wood et al. (1990), J. Immunol. 145:3011-3016). The dihydrofolate reductase (DHFR)-deficient mutant cell lines (Urlaub et al. (1980), Proc Natl Acad Sci USA 77: 4216-4220), DXB11 and DG-44, are desirable CHO host cell lines because the efficient DHFR selectable and amplifiable gene expression system allows high level recombinant protein expression in these cells (Kaufman R. J. (1990), Meth Enzymol 185:537-566). In addition, these cells are easy to manipulate as adherent or suspension cultures and exhibit relatively good genetic stability. CHO cells and proteins recombinantly expressed in them have been extensively characterized and have been approved for use in clinical commercial manufacturing by regulatory agencies.

The methods of the invention can be used to culture cells that express recombinant proteins of interest. The expressed recombinant proteins may be produced intracellularly or be secreted into the culture medium from which they can be recovered and/or collected. In addition, the proteins can be purified, or partially purified, from such culture or component (e.g., from culture medium or cell extracts or bodily fluid) using known processes and products available from commercial vendors. The purified proteins can then be “formulated”, meaning buffer exchanged, sterilized, bulk-packaged, and/or packaged for a final user. Suitable formulations for pharmaceutical compositions include those described in Remington's Pharmaceutical Sciences, 18th ed. 1995, Mack Publishing Company, Easton, Pa.

The present invention is not to be limited in scope by the specific embodiments described herein that are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

In particular preferred embodiments of the present invention, the methods and techniques disclosed herein are used to produce etanercept. Etanercept is a soluble form of the p75 TNF receptor fused to an Fc domain of a human IgG1 (TNFR:Fc). A commercially available etanercept is known as ENBREL®. ENBREL® is produced by recombinant DNA technology in a Chinese hamster ovary (CHO) mammalian cell expression system. It consists of 934 amino acids and has an apparent molecular weight of approximately 150 kilodaltons (Physicians Desk Reference, 2002, Medical Economics Company Inc.). The full sequence expressed in CHO cells is shown below. However, it is to be understood that minor modifications and deletions of this sequence (up to 10%) may be possible and can be used within the scope of the invention. The methods and techniques of the present invention can be used to produce biosimilar etanercept that differs from ENBREL® in, for example, its formulation, dosage, presentation, approved indications or uses, sequence, glycosylation pattern, or other post-translational modification.

```
(SEQ ID NO: 1)
    1 Leu-Pro-Ala-Gln-Val-Ala-Phe-Thr-Pro-Tyr-
  11 Ala-Pro-Glu-Pro-Gly-Ser-Thr-Cys-Arg-Leu-
  21 Arg-Glu-Tyr-Tyr-Asp-Gln-Thr-A1a-Gln-Met-
  31 Cys-Cys-Ser-Lys-Cys-Ser-Pro-Gly-Gln-His-
  41 Ala-Lys-Val-Phe-Cys-Thr-Lys-Thr-Ser-Asp-
  51 Thr-Val-Cys-Asp-Ser-Cys-Glu-Asp-Ser-Thr-
  61 Tyr-Thr-Gln-Leu-Trp-Asn-Trp-Val-Pro-Glu-
  71 Cys-Leu-Ser-Cys-Gly-Ser-Arg-Cys-Ser-Ser-
  81 Asp-Gln-Val-Glu-Thr-Gln-Ala-Cys-Thr-Arg-
  91 Glu-Gln-Asn-Arg-Ile-Cys-Thr-Cys-Arg-Pro-
101 Gly-Trp-Tyr-Cys-Ala-Leu-Ser-Lys-Gln-Glu-
111 Gly-Cys-Arg-Leu-Cys-Ala-Pro-Leu-Arg-Lys-
121 Cys-Arg-Pro-Gly-Phe-Gly-Val-Ala-Arg-Pro-
131 Gly-Thr-Glu-Thr-Ser-Asp-Val-Val-Cys-Lys-
141 Pro-Cys-Ala-Pro-Gly-Thr-Phe-Ser-Asn-Thr-
151 Thr-Ser-Ser-Thr-Asp-Ile-Cys-Arg-Pro-His-
161 Gln-Ile-Cys-Asn-Val-Val-Ala-Ile-Pro-Gly-
171 Asn-Ala-Ser-Met-Asp-Ala-Val-Cys-Thr-Ser-
181 Thr-Ser-Pro-Thr-Arg-Ser-Met-Ala-Pro-Gly-
191 Ala-Val-His-Leu-Pro-Gln-Pro-Val-Ser-Thr-
201 Arg-Ser-Gln-His-Thr-Gln-Pro-Thr-Pro-Glu-
211 Pro-Ser-Thr-Ala-Pro-Ser-Thr-Ser-Phe-Leu-
221 Leu-Pro-Met-Gly-Pro-Ser-Pro-Pro-Ala-Glu-
231 Gly-Ser-Thr-Gly-Asp-Glu-Pro-Lys-Ser-Cys-
241 Asp-Lys-Thr-His-Thr-Cys-Pro-Pro-Cys-Pro-
251 Ala-Pro-Glu-Leu-Leu-Gly-Gly-Pro-Ser-Val-
261 Phe-Leu-Phe-Pro-Pro-Lys-Pro-Lys-Asp-Thr-
271 Leu-Met-Ile-Ser-Arg-Thr-Pro-Glu-Val-Thr-
281 Cys-Val-Val-Val-Asp-Val-Ser-His-Glu-Asp-
291 Pro-Glu-Val-Lys-Phe-Asn-Trp-Tyr-Val-Asp-
301 Gly-Val-Glu-Val-His-Asn-Ala-Lys-Thr-Lys-
311 Pro-Arg-Glu-Glu-Gln-Tyr-Asn-Ser-Thr-Tyr-
321 Arg-Val-Val-Ser-Val-Leu-Thr-Val-Leu-His-
331 Gln-Asp-Trp-Leu-Asn-Gly-Lys-Glu-Tyr-Lys-
341 Cys-Lys-Val-Ser-Asn-Lys-Ala-Leu-Pro-Ala-
351 Pro-Ile-Glu-Lys-Thr-Ile-Ser-Lys-Ala-Lys-
361 Gly-Gln-Pro-Arg-Glu-Pro-Gln-Val-Tyr-Thr-
371 Leu-Pro-Pro-Ser-Arg-Glu-Glu-Met-Thr-Lys-
381 Asn-Gln-Val-Ser-Leu-Thr-Cys-Leu-Val-Lys-
391 Gly-Phe-Tyr-Pro-Ser-Asp-Ile-Ala-Val-Glu-
401 Trp-Glu-Ser-Asn-Gly-Gln-Pro-Glu-Asn-Asn-
411 Tyr-Lys-Thr-Thr-Pro-Pro-Val-Leu-Asp-Ser-
421 Asp-Gly-Ser-Phe-Phe-Leu-Tyr-Ser-Lys-Leu-
431 Thr-Val-Asp-Lys-Ser-Arg-Trp-Gln-Gln-Gly-
441 Asn-Val-Phe-Ser-Cys-Ser-Val-Met-His-Glu-
451 Ala-Leu-His-Asn-His-Tyr-Thr-Gln-Lys-Ser-
461 Leu-Ser-Leu-Ser-Pro-Gly-Lys
```

The methods, techniques, and compositions of the present invention can be used in combination with other methods, techniques, and compositions for producing recombinant protein products, in particular for producing etanercept. Examples of such other methods, techniques, and compositions are found in U.S. Pat. Nos. 7,915,225; 8,119,605; 8,410,060, 8,722,631; 7,648,702; 8,119,604; 8,828,947; 7,294,481; 7,476,722; 7,122,641; 6,872,549; 7,452,695; 7,300,773; 8,063,182; 8,163,522; 6,924,124; 7,645,609; 6,890,736; 7,067,279; 7,384,765; 7,829,309; 6,974,681; 6,309,841; 7,834,162; 8,217,153; 8,273,707; 7,781,395; 7,427,659; 7,157,557; 7,544,784; 7,083,948; 7,723,490; 8,053,236; 7,888,101; 8,247,210; 8,460,896; 8,680,248; 6,413,744; 7,091,004; 6,897,040; 6,632,637; 7,956,160; and 7,276,477, each of which is incorporated by reference in its entirety for all that it teaches or discloses.

### EXAMPLE

This example demonstrates the unexpected finding that temperature in an etanercept N-1 (perfusion) can be controlled to increase titer in the subsequent N (production) bioreactor.

A central composite design (CCD) was used to model the effects of certain operating parameters (seed VCD, culture duration, bioreactor pH and bioreactor temperature) on N-1 perfusion bioreactor performance (i.e. cell growth and viability at the end of the N-1 step). The N-1 bioreactors were also forward-processed into a production bioreactor to assess the impact of these parameters on titer.

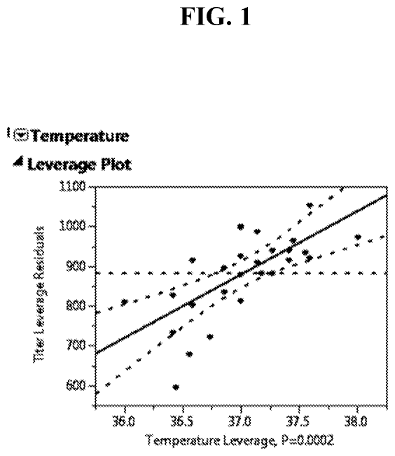

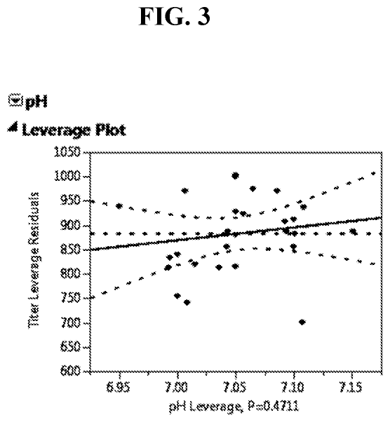

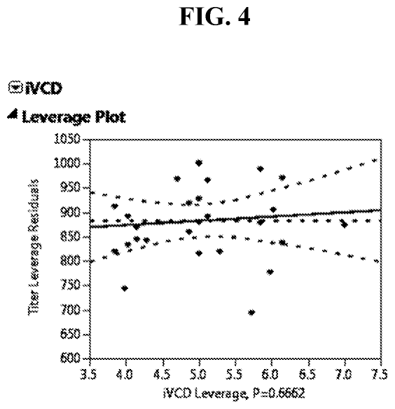

A CCD is a type of response surface design appropriate for estimating a full quadratic model, including both linear, two-factor interactions, and quadratic effects of the OP. This experimental design was chosen based on its structure and statistical properties. A CCD contains a 2^{k} factorial design with center points that are augmented with a group of axial points that allow the estimation of curvature. By choosing the OR for the factorial points, all combinations can be directly observed in the experiment. In addition, by setting the axial points to the desired characterized ranges, a model can be developed to conduct sensitivity analysis at different combinations of these values. Results are provided in Table 1 and FIGS. 1 through 4.

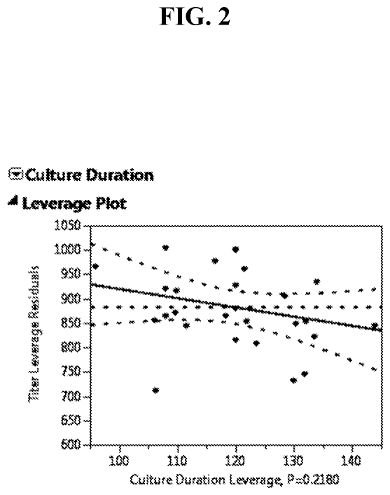

As shown in Table 1 and FIG. 1, the data demonstrate that an increase in the N-1 bioreactor temperature, within the range tested, resulted in a statistically significant and reproducible increase in production bioreactor titer despite the production bioreactors being run at center point (that is, the production bioreactors were run using the same conditions, including temperature, pH, culture duration, and N iVCD). In contrast, FIGS. 2-4 show that no statistically significant increase in titer was observed when the other tested parameters (pH, duration and N-1 iVCD) were altered within the tested parameters. (Data for all figures are provided in Table 1.)

TABLE 1

| | Culture | | | | |
|---|---|---|---|---|---|
| | Duration | pH | Temperature | iVC | Titer |
| 1 | 108 | 7 | 37.5 | 4 | 930 |
| 2 | 108 | 7 | 36.5 | 6 | 917 |
| 3 | 132 | 7.1 | 36.5 | 6 | 786 |
| 4 | 132 | 7.1 | 37.5 | 4 | 934 |
| 5 | 132 | 7 | 37.5 | 6 | NT |
| 6 | 108 | 7.1 | 37.5 | 4 | 1003 |
| 7 | 108 | 7.1 | 37.5 | 6 | 1018 |
| 8 | 120 | 7.05 | 37 | 5 | 996 |
| 9 | 132 | 7 | 36.5 | 4 | 625 |
| 10 | 120 | 7.05 | 37 | 5 | 925 |
| 11 | 120 | 7.05 | 37 | 5 | 812 |
| 12 | 108 | 7 | 37.5 | 6 | 923 |
| 13 | 108 | 7 | 36.5 | 4 | 748 |
| 14 | 120 | 7.05 | 37 | 5 | 877 |
| 15 | 132 | 7.1 | 37.5 | 6 | 1036 |
| 16 | 108 | 7.1 | 36.5 | 6 | 648 |
| 17 | 132 | 7.1 | 36.5 | 4 | 821 |
| 18 | 108 | 7.1 | 37.5 | 4 | 946 |
| 19 | 132 | 7 | 37.5 | 4 | 880 |
| 20 | 132 | 7 | 36.5 | 6 | 658 |
| 21 | 120 | 7.15 | 37 | 5 | 884 |
| 22 | 120 | 7.05 | 37 | 5 | 998 |
| 23 | 120 | 7.05 | 37 | 5 | 996 |
| 24 | 120 | 7.05 | 36 | 5 | 809 |
| 25 | 96 | 7.05 | 37 | 5 | 963 |
| 26 | 144 | 7.05 | 37 | 5 | 841 |
| 27 | 120 | 7.05 | 38 | 5 | 972 |
| 28 | 120 | 6.95 | 37 | 5 | 936 |
| 29 | 120 | 7.05 | 37 | 7 | 871 |
| 30 | | 7.05 | 37 | 3 | 973 |

1. A method of producing etanercept from recombinant CHO cells comprising running an N-1 perfusion bioreactor at a temperature of between 37° C. to 38° C. before running an N production bioreactor.

2. The method of claim 1, wherein said N-1 perfusion bioreactor is run at a temperature of 37.5° C.

3. The method of claim 1, wherein said N-1 perfusion bioreactor and said N production bioreactor are run in different bioreactor vessels.

4. The method of claim 1, wherein said N-1 perfusion bioreactor and said N production bioreactor are run in the same bioreactor vessel.

5. The method of claim 1, wherein said method produces an N production bioreactor titer that is higher than the N production bioreactor titer of an otherwise identical method of producing etanercept whose N-1 perfusion bioreactor is run at a temperature below 37° C.

6. The method of claim 2, wherein said method produces an N production bioreactor titer that is higher than the N production bioreactor titer of an otherwise identical method of producing etanercept whose N-1 perfusion bioreactor is run at a temperature below 37.5° C.

7. The method of claim 1, wherein the total protein produced by said N production bioreactor is higher than the total protein produced by the N production bioreactor of a method of producing etanercept whose N-1 perfusion bioreactor is run at a temperature below 37° but is otherwise identical.

8. The method of claim 2, wherein the total protein produced by said N production bioreactor is higher than the total protein produced by the N production bioreactor of a method of producing etanercept whose N-1 perfusion bioreactor is run at a temperature below 37.5° C. but is otherwise identical.

9. A formulation of etanercept comprising etanercept produced using the method of claim 1.

10. The formulation of claim 9, wherein said formulation consists of 50 mg/mL of said etanercept, 1% sucrose (w/v), 100 mM sodium chloride, 25 mM sodium phosphate, 25 mM L-arginine, and water.

11. The formulation of claim 9, wherein said formulation consists of 25 mg of said etanercept, 40 mg mannitol, 10 mg sucrose, and 1.2 mg tromethamine in a lyophilized powder.

12. The formulation of claim 9, wherein said formulation consists of 50 mg/mL of said etanercept, 1% sucrose (w/v), 120 mM sodium chloride, 25 mL L-arginine, and water.

13. The formulation of claim 10, wherein 1 mL of said formulation is in a prefilled syringe or SURECLICK® autoinjector.

14. The formulation of claim 10, wherein 0.51 mL of said formulation is in a prefilled syringe or SURECLICK® autoinjector.

15. The formulation of claim 11, wherein said lyophilized powder is in a multiple-use vial.

16. A method of treating a patient in need thereof, comprising administering to said patient an effective dose of etanercept produced using the method of claim 1.

17. The method of claim 10 wherein said patient has rheumatoid arthritis, polyarticular juvenile idiopathic arthritis, psoriatic arthritis, ankylosing spondylitis, or plaque psoriasis.

18. A kit comprising the formulation of claim 9 and instructions for use of said formulation.

19. The formulation of claim 12, wherein 1 mL of said formulation is in a prefilled syringe or SURECLICK® autoinjector.

20. The formulation of claim 12, wherein 0.51 mL of said formulation is in a prefilled syringe or SURECLICK® autoinjector.
